(19) United States
(12) Patent Application Publication
(10) Pub. No.: US 2012/0289821 A1
(43) Pub. Date: Nov. 15, 2012

(54) C-ARM INTEGRATED ELECTROMAGNETIC TRACKING SYSTEM

(75) Inventors: Rainer Graumann, Hochstadt (DE); Gerhard Kleinszig, Erlangen (DE); Jeffrey Siewerdsen, Baltimore, MD (US); Jongheun Yoo, Baltimore, MD (US)

(73) Assignees: THE JOHNS HOPKINS UNIVERSITY, Baltimore, MD (US); SIEMENS AKTIENGESELLSCHAFT, Muenchen (DE)

(21) Appl. No.: 13/304,951

(22) Filed: Nov. 28, 2011

Related U.S. Application Data
(60) Provisional application No. 61/485,587, filed on May 12, 2011.

(51) Int. Cl. A61B 6/00; H05G 1/64; A61B 6/03

(52) U.S. Cl. 600/424; 378/19; 378/98; 378/8

(57) ABSTRACT

A medical imaging system and a method electromagnetically track a position of structures with the medical imaging system and a C-arm arrangement. The medical imaging system contains a C-arm, a gantry, and at least one electromagnetic field generator assembly with at least one electromagnetic field generator which interacts with an electromagnetic sensor from receiving the electromagnetic radiation. Preferably, the electromagnetic sensor is positioned within a region of surgical interest in a patient. The electromagnetic field generator is directly embedded into the C-arm.

### CROSS-REFERENCE TO RELATED APPLICATION

This application claims the priority, under 35 U.S.C. §119(e), of provisional application No. 61/485,587 filed May 12, 2011; the prior application is here-with incorporated by reference in its entirety.

### BACKGROUND OF THE INVENTION

### Field of the Invention

The present invention generally relates to medical imaging systems, and more specifically, to an electromagnetic tracking system for use within an imaging device.

Image-guided surgical or interventional procedures are often based on (2D or 3D) positional data of medical structures, surgical instruments, or implants (for example for surgical instruments or an anatomical structure of the patient etc.). Therefore it is necessary that these structures, for example surgical instruments and the like, that are used during this medical procedure are tracked, so that their position may be superimposed onto an image, acquired by the modality in order to track the relative position of the surgical instrument with respect to the region of interest in the patient's body.

Generally, there exist different types of tracking methods, like optical tracking, electromagnetic tracking and image based tracking. The present invention relates to an electromagnetic tracking.

Within imaging technology different imaging technologies exist, such as computed tomography (CT), magnetic resonance imaging (MRI), C-arm fluoroscopic imaging or two or three dimensional fluoroscopes or ultrasound imaging (US) as well as medical linear accelerator techniques for radiation therapy. The present invention improves electromagnetic surgical tracking in combination with a C-arm.

Up to now known medical procedures use common (mainly: mobile) C-arms and combine the C-arm device with an electromagnetic tracking system as a separate system.

Conventional systems are set-up in that electromagnetic field generators are mounted on a separate device, mainly on an arm on tableside. This conventional set-up is also depicted in FIG. 1, in which one electromagnetic field generator is placed on a support structure (a tripod or articulated arm) at tableside such that the electromagnetic tracker field of view includes the region of surgical interest.

For example the “Aurora” system of the company Northern Digital Inc. (NDI), Canada, may be used which is designed for applications requiring precise medical measurements.

However, the conventional set-up involves a number of limitations. First, arranging essential components of the electromagnetic tracking system is time-consuming and adds complexity to the operating room set-ups. Equipment associated with the conventional set-up includes for example an electromagnetic field generator, a tracker control unit, wired tracked tools, a plurality of power-cables (at least one for the control unit, for the field generator and possibly for the electromagnetic sensors) and a serial communication cable to a computer. In practice a drawback is to be seen in that the use of image guided surgical systems is often limited due to the cables, the field generator support structure and the field generator itself. This is especially important in x-ray-guided interventions, where each of the above mentioned structures of equipment can occlude the x-ray-beam.

Another drawback is the position of the field generator beside and over the operating-table because size and dimensions of field generators used may be in the area of around 200 mm×200 mm×70 mm and therefore may occlude a part of the patient to be examined. Additionally, this known field generator arrangement may inhibit prompt action on the part of the surgical staff in responding to a medical emergency health situation. A main disadvantage of the current set-up is, that a registration procedure has to be applied each time anew in order to calculate transformation matrix between imaging modality and field generator. Present invention overcomes this drawback.

Finally, the conventional set-up requires sterile tracking.

### SUMMARY OF THE INVENTION

It is, therefore, desirable to provide an improved electromagnetic tracking system with a surgical C-arm. Particularly, the C-arm or the gantry of a medical imaging system should be improved in that known further equipment and tracking structures are necessary before tracking purposes. A further object of the present invention is to provide a combined imaging and tracking device without the need to provide separate cables and interfaces for the tracking system.

In accordance with the teachings of the present invention, a medical imaging system contains a C-arm with an x-ray-detector and an x-ray-emitter, a gantry for supporting and supplying at least the C-arm, and at least one electromagnetic field generator assembly for emitting electromagnetic radiation for use within an electromagnetic tracking system. According to the invention the field generator assembly is (optionally: removably) embedded into the C-arm.

In contrast to known tracking set-up's, which require that each time a new registration procedure has to be executed for computing a transformation matrix between imaging modality and field generator assembly. With the present invention the burdensome repetition of the registration procedure can be avoided by a fixed spatial relationship between the field generator assembly and imaging modality (e.g. with an integrated system).

In an embodiment of the invention, at least a part of the electromagnetic tracking system is directly implemented in the surgical C-arm or in the gantry of the medical imaging system in order to track the patient, an anatomically structure of the patient, patient support and/or various trackable tools (instruments, chirurgical structures etc.). An important aspect in combining an electromagnetic tracking system with a C-arm is the fixed registration of the C-arm with the tracking system.

In another embodiment, the electromagnetic field generator assembly is a frame-like or window-like field generator, which is positioned on the C-arm such that the opened portion of the frame-like field generator is about the x-ray-detector of the medical imaging system, which could for example be a flat-detector or an x-ray-image intensifier.

In yet another embodiment a single electromagnetic field generator is provided. In alternative embodiments a plurality of electromagnetic field generators may be provided.

Further, the electromagnetic field generator may be incorporated directly on the C-arm gantry. The field generator may be positioned on the C-arm near the midpoint of the C-shaped gantry. Alternatively, the field generator may be positioned at another position on the C-arm gantry, for example more narrowly to the x-ray-tube of the C-arm.

Another embodiment relates to providing a plurality of electromagnetic field generators which all are directly incorporated and embedded on/in the C-arm gantry.

Yet another embodiment refers to providing an array of electromagnetic coils. The term “coil” refers to the component that generates the electromagnetic field of the electromagnetic tracking system. In this embodiment an array of coils is directly integrated within the gantry such that this C-arm itself is essentially the field generator. Another embodiment may employ coils as receivers and transponders for the electromagnetic signals, sent by the field generator assembly.

The present invention further relates to a method for electromagnetically tracking the position of a (patient or surgical tool) structure to be examined with a medical imaging system, containing a C-arm with an x-ray-detector and an x-ray-emitter, supported by a gantry and containing at least one electromagnetic field generator assembly for emitting electromagnetic radiation to be used in an electromagnetic tracking system or to be received by an electromagnetic sensor assembly. The electromagnetic field generator assembly is removable or permanently embedded into the C-arm. The method includes the following steps: providing electromagnetic sensors at the structure (there also could be several structures), which position should be tracked, positioning the patient with the structure in the medical imaging system, particularly on the table of the medical imaging system, and activating the at least one electromagnetic field generator assembly for emitting electromagnetic radiation to be received by at least one electromagnetic sensor inside the structure.

The local magnetic field strength (intensity) is measured. A position of each sensor is calculated by providing a position tracking with configurable degrees of freedom (for example five or six degrees of freedom).

Another aspect of the present invention refers to a C-arm assembly of a medical imaging system, wherein at least one electromagnetic field generator assembly for emitting electromagnetic radiation for use within an electromagnetic tracking system is (removable or permanently fixed) embedded or integrated into the C-arm.

Referring to the technical realization of the integration of field generators into the C-arm the following embodiments might be used. In order to avoid interferences between the electromagnetic field generated by the field generator(s) and the ferro-magnetic material of the C-arm, the field generator can be shielded against the C-arm by using a shielding, like for example at least one plate of aluminum between field generator and C-arm. In case the field generator/s is/are fixedly mounted to the C-arm, the affects of the ferromagnetic material of the C-arm onto the electromagnetic field is constant and can be calibrated once.

Other features which are considered as characteristic for the invention are set forth in the appended claims.

Although the invention is illustrated and described herein as embodied in a C-arm integrated electromagnetic tracking system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of a conventional magnetic tracking system, involving a field generator as a separate structure on a support arm at tableside according to the prior art systems;

FIG. 2 is a diagrammatic, perspective view of a first embodiment of a frame-shaped field generator assembly according to the invention;

FIG. 3 is a diagrammatic, perspective view of a second embodiment according to the invention with a single field generator mounted in a middle of a C-arm gantry;

FIG. 4 is a diagrammatic, perspective view of a third embodiment showing multiple field generators, each being embedded into the C-arm gantry according to the invention; and

FIG. 5 is a diagrammatic, perspective view of a fourth embodiment of an array of electromagnetic coils, which are integrated within the C-arm gantry, according to the invention.

### DETAILED DESCRIPTION OF THE INVENTION

The following description of the preferred embodiments is nearly exemplary in nature and is in no way intended to limit the invention, its application, or uses. Moreover, while the invention is discussed in detail below with regard to a mobile C-arm, the field generator assembly according to the present invention may be used with any type of medical x-ray-modality and also with other types of C-arms, including fixed-room C-arms in interventional radiology and/or surgery (for example ceiling-mounted or floor-mounted devices), robotic imaging systems, CT-scanners, medical linear accelerators for radiation therapy or other types of robotic linear accelerators.

In the following there is given a short explication and definition of term, used within this patent application.

The term “embedded” is to be construed such that the electromagnetic field generator assembly is directly integrated into the C-arm. According to first embodiment the electromagnetic field generator assembly is removable integrated or embedded into the C-arm. According to a second embodiment the electromagnetic field generator assembly is permanently integrated in the C-arm, for example with screws, bolts or pins. The second embodiment has the advantage and the further feature that the electromagnetic field generator may also be used for balancing the weight of the C-arm, so that there is a fixed weight balance for the C-arm with the integrated field generator assembly.

It has to be mentioned that according to the invention the electromagnetic field generator assembly is not attached to the C-arm as a separate device, but is provided integrally with the C-arm assembly. According to an embodiment of the present invention the electromagnetic field generator assembly is formed monolithically with the C-arm or with the gantry of the C-arm. In this embodiment it is not possible to separate the field generator from the imaging device. According to another embodiment the electromagnetic field generator assembly is fixed in its relative position to the C-arm or to the gantry. Thus, the position of the electromagnetic field generator assembly may not be changed independently of the position of the C-arm or of the gantry. Yet, a further embodiment refers to an electromagnetic field generator assembly, which is positioned within the radiation area of the x-ray-beam. Another embodiment refers to the fact that the position of the electromagnetic field generator assembly may only be changed by changing the position of the C-arm or of the gantry. Yet, another embodiment refers to the fact that the field generator assembly has a constant distance from the C-arm or the gantry of the same. Thus, it is not traversable or rotatable attached to the C-arm. Moreover, the electromagnetic field generator is integrated or inbuilt into the C-arm or in the gantry of the same. According to a preferred embodiment the electromagnetic field generator is integral with a C-arm or gantry. The electromagnetic field generator could also belong to the same sales unit as the C-arm or as the gantry.

According to a preferred embodiment the electromagnetic tracking system (with the electromagnetic field generator assembly) and the C-arm are consolidated in a common tracking-C-arm-system. Moreover, the tracking system and the C-Arm may be operated in common and/or may use the same support structure and/or power source and/or interfaces.

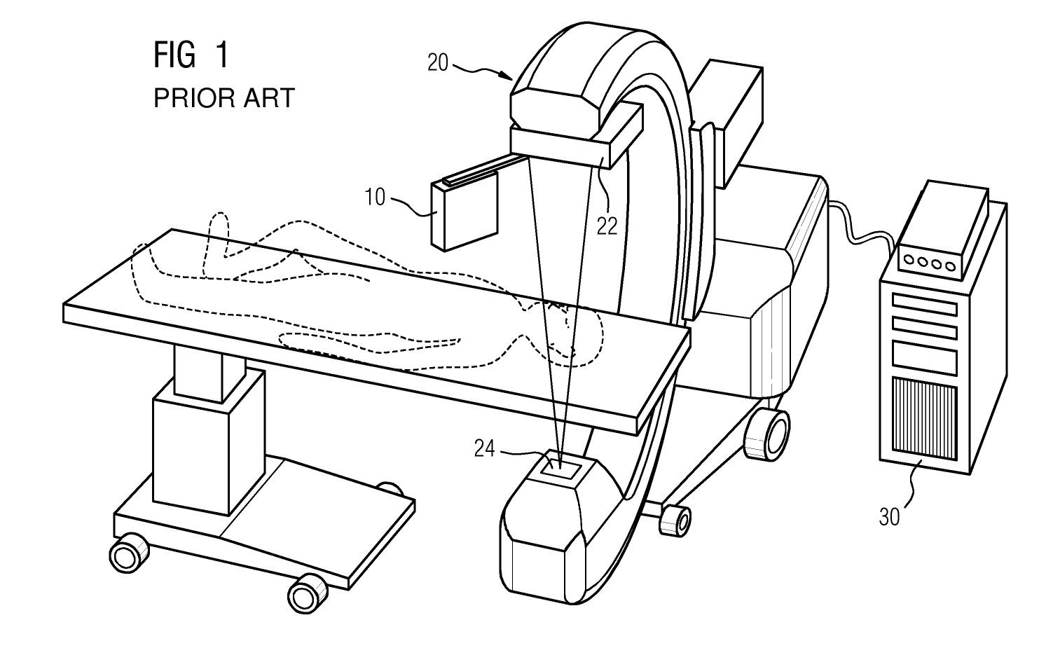

FIG. 1 shows a schematic drawing of a known field generator 10 of a known set-up for an electromagnetic tracking system. As can be seen in FIG. 1 the field generator is mounted on a separate tripod, which is connected by wire to a C-arm 20. As may be seen in FIG. 1 the conventional electromagnetic tracker set-up involves a field generation on a separate support arm at tableside, which may be positioned with respect to a region of surgical interest. This conventional set-up involves power cables for the electromagnetic tracking control unit and for the electromagnetic field generator as well as serial communication cables to a computer, and wired tracked tools (normally positioned within a patient's structure to be examined).

In contrast to this, the present invention provides the electromagnetic field generator assembly 10, being directly integrated into the C-arm 20.

The incorporation of the electromagnetic field generator assembly is referred to as “on-board-configuration”. Although the field generator 10 is within a reference frame that is potentially changing continuously (i.e. gantry rotation) registration of the system can be maintained by a reference marker within the field of view.

The field generator assembly according to the invention may be used as an electromagnetic navigation system for interventional procedures. The navigation system contains at least one electromagnetic field generator assembly for emitting an electromagnetic field and at least one field sensor assembly for receiving the electromagnetic field. Preferably, the electromagnetic field generator assembly is directly embedded into the C-arm 20. The electromagnetic sensor assembly is integrated in structures, which position should be tracked or which volume should be navigated (anatomical patient's structures, medical instruments etc.). The generator assembly is controlled by a control unit 30.

The electromagnetic spatial measurement and navigation system according to the invention determines the location of structures or objects that are embedded with sensor coils or a sensor assembly. When the object is placed inside controlled, varying magnetic fields, voltages are induced in the sensor coils. These induced voltages are used by the measurement system to calculate the position and orientation of the object. Based on the received magnetic field at the sensor assembly voltage is induced dependent on the position of the sensor assembly (distance between field sensor assembly and the field generator). As the magnetic fields are of low field strength and can safely pass through human tissue, location measurement of an object is possible without the line-of-sight constraints of an optical spatial measurement system.

The control unit 30 applies a potential (voltage) so that the generator assembly generates a magnetic field which is sufficiently high and homogeneous to be received by the sensor assembly. The control unit 30 is also adapted to activate the field generator assembly in a time-based manner (for example in configurable time intervals).

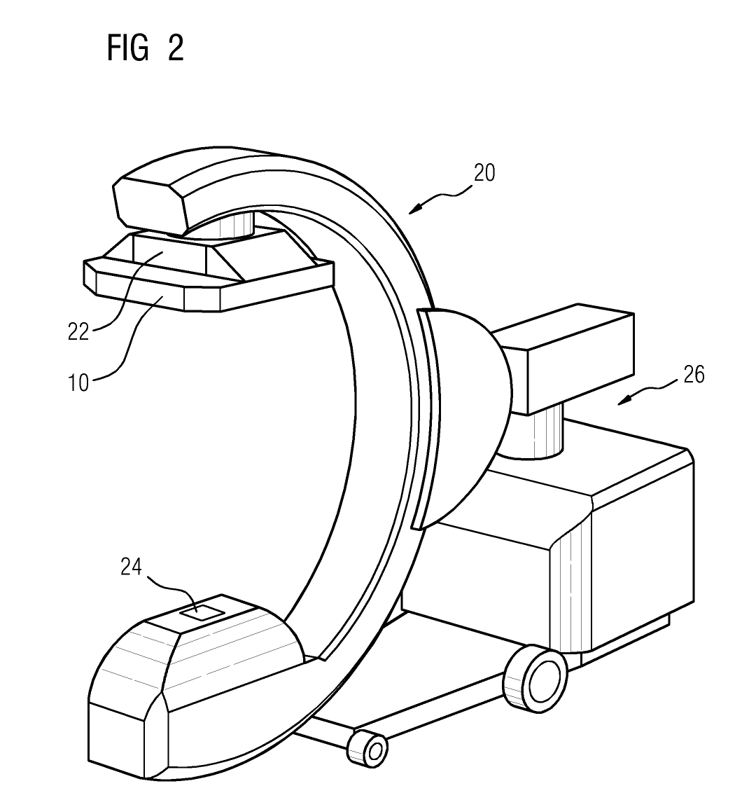

One embodiment of the invention is depicted in FIG. 2. Here, the electromagnetic field generator 10 is provided as a frame structure, particularly as a ring-like or rectangular frame. The generator 10 is integrated into the C-arm 20 at the upper side of the C-arm, near by the flat panel detector 22. Normally, the generator 10 is positioned on the C-arm 20 such that the opened portion of the detector 22 is fully or at least partially surrounded by the generator 10. A gantry 26 allows for rotating the C-arm 20 in different positions in order to scan the patient in several C-arm angles and in several projections. Usually, the C-arm 20 is a mobile C-arm for cone-beam computer tomography. In this embodiment the placement allows for electromagnetic shielding to be incorporated about the field generator without interfering with the x-ray-beam.

According to an aspect of the present invention the electromagnetic tracking control unit 30 would be optionally integrated within the C-arm 20, too. According to an alternate embodiment the control unit 30 may be provided as a separate device, which may be linked to the tracking system by a respective interface (wired or wireless, like Bluetooth etc.).

Another aspect is directed to the fact that the control unit 30 may share a power source and communication to the computer with the C-arm 20. In this case no separate supporting and supply system for the control unit 30 would be necessary.

With respect to the embodiment, shown FIG. 2, the frame-shaped field generator 10 is completely x-ray-compatible. Thus, the field generator 10 does not occlude the x-ray-beam and allows normal radiographic, fluoroscopic, or cone-beam CT x-ray-imaging. In the embodiment, shown in FIG. 2, the window-shaped field generator 10 is mounted, particularly directly assembled, on the C-arm 20 about the x-ray-detector 22, which could be a flat-panel detector 22.

On the left hand side of FIG. 2 an overview of one example embodiment is given, whereas on the right hand side of FIG. 2 four examples, depicted in smaller images, illustrate the electromagnetic field generator assembly field of view, showing that the electromagnetic tracking maintains the region of surgical interest within its field of view for all angulations of the C-arm 20. Thus, on the right hand side the window-shaped field generator 10 is depicted at various C-arm-angles. The upper most image relates to an angle of −45 Grad, the second image relates to an angle of 0 Grad, the third image relates to an angle of +45 Grad and the fourth, last bottom most image relates to an angle of +90 Grad.

According to another example embodiment only one field generator is used in the field generator assembly. Other embodiments relate to using more than one field generator 10 in the field generator assembly.

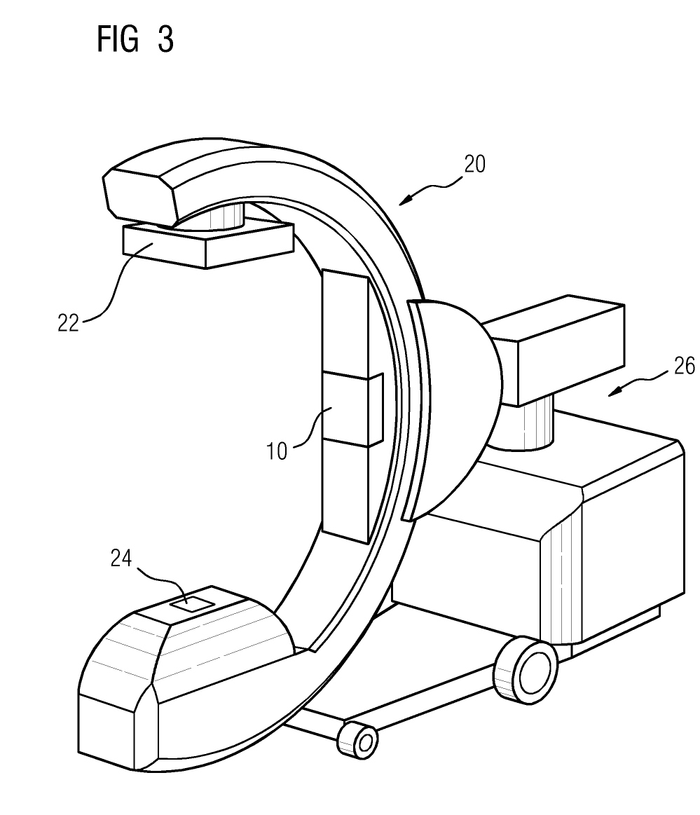

FIG. 3 also shows two sections (like FIG. 2), wherein on the left hand side a schematic overview is depicted and on the right hand side the field generator 10 is depicted at various C-arm-angles.

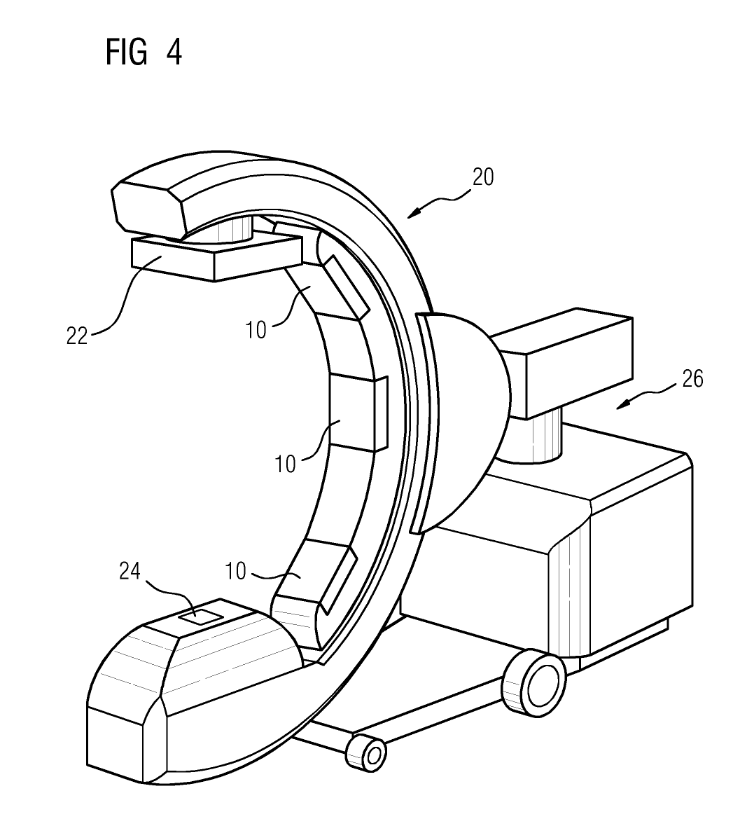

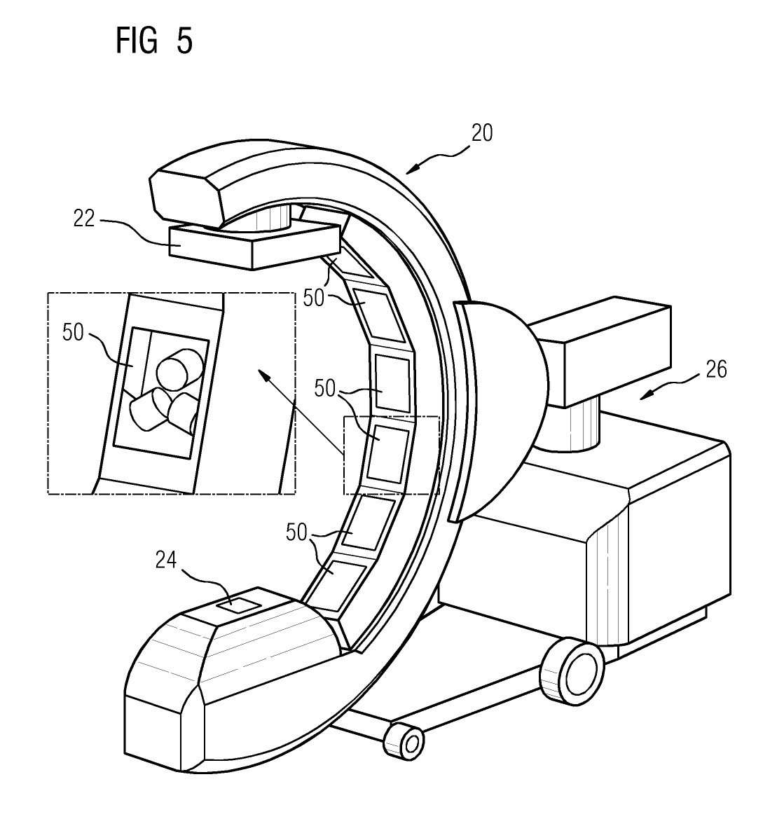

According to the specific embodiment the field generator 10, named “Aurora” from the company NDI, Waterloo, Canada, is used. In this example embodiment one single electromagnetic field generator 10 is incorporated directly on the C-arm gantry 26. In this case, the field generator 10 is positioned on the C-arm 20 near the midpoint of the C-shaped gantry 26. Principally, the field generator 10 could be placed anywhere on the gantry 26 (i.e. more close to the detector 22 or more close to the x-ray-tube 24). Preferably, the electromagnetic tracker control unit 30, with cables etc. would optionally be integrated within the C-arm 20, too. This option (of integrating the control unit 30 into the C-arm 20 may also be applied in all the other example embodiments, mentioned in this application (and also below with respect to FIGS. 4 and 5). As already mentioned with respect to FIG. 2 on the right hand side the field generator assembly on the C-arm 20 is shown at various C-arm-angles. For the description of the smaller images with respect to the different angles it is referred to the description of FIG. 2 which might be applied also to FIG. 3.

FIG. 4 relates to an embodiment where multiple field generators 10 are used for being embedded or integrated in the C-arm 20. In this embodiment a plurality of field generators 10 is directly incorporated on the C-arm gantry 26. In other respects, the embodiment is similar to that embodiment described above with respect to FIG. 3, except that it includes a plurality of field generators 10. The tracker field of view is potentially larger and better includes the region of surgical interest at various C-arm-angulations. Again in FIG. 4 on the right hand side there are depicted multiple field generators 10 at various C-arm angles, which are mounted on the C-arm gantry 26.

A further example embodiment is described with respect to FIG. 5. This embodiment refers to an integrated array of field generator coils 50. As may be seen in FIG. 5, an array of electromagnetic coils 50 is incorporated on the C-arm gantry 26. The coils 50 relate to the components that generate the electromagnetic field. This embodiment is distinct from the previous examples in several aspects. First, it does not use a fixed configuration of (an “off-the-shelf”) electromagnetic field generator 10. By contrast, it integrates an array of coils 50 within the gantry 26 such that the C-arm 20 itself is essentially the field generator. Moreover, it provides potentially larger field of view and greater tracking precision by virtue of a large distributed array of coils 50 that may be more freely optimized than in a constrained footprint of a conventional field generator package, as mentioned in the embodiments before. FIG. 5 shows three sections. On the left hand side in the upper image a schematic overview is given in which an array of electromagnetic coils 50 are integrated within the C-arm gantry 26 to form an electromagnetic field generator within the entire C-arm envelope.

The lower image on the left hand side shows the same configuration as above with an enlarged section of an electromagnetic coil 50. On the right hand side the field generator array concept is depicted at multiple C-arm angles as mentioned before.

According to a further embodiment the control unit 30 may be integrated in the gantry 26, in a trolley or in a chassis of the C-arm 20, which is preferred, because the control unit 30 is as close as possible to the surgical region.

According to the preferred embodiment the field generator assembly, the field generators 10 and/or the coils 50 are statically integrated (in a fixed position) in the C-arm 20. This has the advantage that there is a direct spatial relation between the acquired x-ray image and the position of the sensor coil in the patient's structure. The position of the sensing coil is known in the coordinate system of the C-arm 20. Generally, the sensing coils for receiving the electromagnetic field are placed in a patient's structure or in patient's support or in surgical tools like medical needles or (for the purpose of positioning) in markers, which are fixed in a patient's bone, for supporting bone-growth after bone-fractures.

In case the imaging modality is a CT-device, it is necessary, to make sure, that the field generator 10 does not lay within the active area for the CT-imaging. The field generator 10 may be positioned at both openings of the CT-gantry 26.

In case the field generator assembly is removable integrated into the gantry 26, the invention provides a fastening mechanism. The fastening mechanism makes sure that the field generator 10 may only be fixed in one static position within the gantry 26. Thus, it is not possible, to reposition the field generator 10 into different positions in the gantry 26. Thus, it can be assured that the coordinate transformation between imaging and tracking system is always the same (also only with one and the same registration procedure for several examinations).

In contrast to conventional tracking systems, in which the field generator 10 or the assembly of field generators 10 is positioned on a separate arm or separate support structure at tableside, the invention characterizes in that the field generator(s) 10 is/are directly in-built into the gantry 26 without a separate supply structure.

According to an embodiment of present invention there is provided a shielding. The shielding might be a metal plate, like an aluminum plate, for example in 2-3 millimeter thickness. The shielding is provided in order to protect the field generator assembly and/or the sensor assembly against disturbance or interference based on ferromagnetic material (which might in the end lead to positioning failures). In order to minimize this effect, the shielding is provided between the field generator (assembly) and the C-arm. Although the shielding also interferes with the electromagnetic field of the electromagnetic field generator assembly, this interference is well defined and therefore the modifications based thereon are predictable and calculable, so that the effect may be considered for positioning and tracking.

Another possibility is that the electromagnetic field generator and sensor assemblies each have to separated from ferromagnetic materials or they are made of a specific steel (for surgical purpose) with a minimum of ferromagnetic properties.

The inventions shows several significant advantages over prior art. The registration procedure may be maintained by means of a reference marker. Moreover, the registration procedure might only take place once, as there is a fixed transformation matrix between tracking system and C-arm. With this approach it is possible to have more flexible configurations of the electromagnetic field generators 10 or the coils 50 on the gantry 26, which in the end provides a potentially increased field of view and accuracy. The electromagnetic field generator assembly, the field generators 10 and/or the coils 50 are positioned in the same frame of reference for the purpose of tracking the position of the patient, a patient's structure, a support table and/or various trackable interventional tools. With the invention, the tracker set-up may be simplified compared to conventional arrangements in which the field generator 10 is mounted on a separate or extra arm positioned at tableside.

Moreover, inter-operative imaging can be performed without disrupting the surgical set-up. A conventional tracker needs to be situated such that it does not block the x-ray beam in radiographic, fluoroscopic, or tomographic use, but the on-board field generator configuration ensures that field generator 10 does not occlude the x-ray beam. The on-board configuration according to the invention allows a higher flexibility with respect to the number and/or positioning/orientation of the field generator 10 and/or of coils 50. Moreover, the on-board configuration has an advantage with respect to sterility, since the tracker may be incased within the gantry 26 rather than placed over the patient (in a sterile bag) as in the conventional set-up.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A medical imaging system, comprising:
a C-arm having an x-ray detector and an x-ray emitter;
a gantry for supporting and supplying said C-arm;
a sensor assembly; and
at least one electromagnetic field generator assembly for emitting electromagnetic radiation to be received by said sensor assembly, said electromagnetic field generator assembly is directly embedded into said C-arm.

2. The medical imaging system according to claim 1, wherein the medical imaging system is selected from the group consisting of an x-ray system and a computer tomography system for use in image-guided surgical or interventional procedures.

3. The medical imaging system according to claim 1, wherein said electromagnetic field generator assembly is a window-shaped frame structure which is in-built at an open portion on said x-ray detector.

4. The medical imaging system according to claim 1, wherein said electromagnetic field generator assembly is directly embedded in said C-arm near a midpoint of a c-shaped c-arm.

5. The medical imaging system according to claim 1, wherein said electromagnetic field generator assembly contains a plurality of field generators, each of said field generators is directly incorporated into said C-arm.

6. The medical imaging system according to claim 1, wherein said electromagnetic field generator assembly contains distributed separate electromagnetic coils, each of said electromagnetic coils is directly embedded into said C-arm, so that said electromagnetic coils generate an electromagnetic field within an entire envelope of said C-arm.

7. The medical imaging system according to claim 1, wherein said sensor assembly contains at least one coil, said coil being placed within a patient to be examined with the medical imaging system.

8. The medical imaging system according to claim 1, further comprising an electromagnetic tracking system having a control unit, said control unit being directly integrated in said C-arm, being integrated in a separate trolley structure or in said gantry of said C-arm.

9. The medical imaging system according to claim 1, further comprising a shielding, at least one of said electromagnetic field generator assembly or said sensor assembly is shielded against said C-arm by using said shielding.

10. The medical imaging system according to claim 1, wherein an effect of ferromagnetic material with regard to an electromagnetic field of said electromagnetic field generator assembly is constant and can be calibrated.

11. The medical imaging system according to claim 3, wherein said window-shaped frame structure is selected from the group consisting of a rectangular frame structure and a circular frame structure.

12. A method for electromagnetically tracking a position of a medical structure during an x-ray procedure, using a medical imaging system containing a C-arm having an x-ray-detector and an x-ray-emitter, and at least one electromagnetic field generator assembly for emitting electromagnetic radiation, the electromagnetic field generator assembly being removable from or permanently embedded into the C-arm, which method comprises the steps of:
providing at least one electromagnetic sensor to the medical structure;
positioning the medical structure having the at least one electromagnetic sensor in the medical imaging system;
activating the at least one electromagnetic field generator assembly for emitting electromagnetic radiation to be received by the at least one electromagnetic sensor inside the medical structure;
measuring a local magnetic field strength at the electromagnetic sensor;
calculating a position of the at least one electromagnetic sensor based on a measured local magnetic field strength; and
tracking a position of the medical structure.

13. A C-arm of a medical imaging system, comprising:
an x-ray detector;
an x-ray emitter;
a C-arm body;
a gantry for supporting and supplying said C-arm body; and
at least one electromagnetic field generator assembly for emitting electromagnetic radiation, the electromagnetic field generator assembly is directly embedded into said C-arm body.
